(19) United States
(12) Patent Application Publication
(10) Pub. No.: US 2002/0010201 A1
(43) Pub. Date: Jan. 24, 2002

(54) Method for treating restless leg syndrome using pramipexole and clonidine

(76) Inventor: Hans Michael Brecht, Ingelheim (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 09/970,839

(22) Filed: Oct. 4, 2001

Related U.S. Application Data
(63) Continuation of application No. 09/640,098, filed on Aug. 15, 2000.

(30) Foreign Application Priority Data
Aug. 19, 1999 (DE) 199 38 825.3

(51) Int. Cl. A61K 31/415

(52) U.S. Cl. 514/388

(57) ABSTRACT

The invention relates to an active substance combination consisting of clonidine and pramipexole for treating Restless Leg Syndrome.

### RELATED APPLICATION

This application is a continuation of Ser. No. 09/640,098, filed Aug. 15, 2000.

### FIELD OF THE INVENTION

The invention relates to a method for treating Restless Leg Syndrome comprising the administration of pramipexole and clonidine, and a pharmaceutical composition suitable for the treatment of Restless Leg Syndrome comprising both pramipexole and clonidine.

### BACKGROUND TO THE INVENTION

Restless Legs Syndrome is a neurological disorder which manifests itself chiefly as sensory disorders of the legs such as tingling, dragging, tearing, itching, burning, cramp or pain and in those affected triggers an irresistible compulsion to move. Frequently these disorders occur when the affected person is resting. Particularly at night, during sleep, these sensory disorders and the consequent compulsive movements lead to restlessness and sleep disorders.

RLS occurs at all ages, increasing in frequency at more advanced ages. The prevalence in the general population is about 5%. Because of the characteristics of the symptoms RLS is one of the most common causes of sleep problems. RLS is the cause of sleeping and waking problems in 7% of 20-40 year-olds, 18% of 40-60 year-olds and 33% of over 60s.

When the patient's quality of sleep or life is increasingly affected by RLS or the patients suffer from daytime tiredness, treatment is indicated. The need for treatment generally sets in at the age of 40-50.

Hitherto there has been no permitted drug treatment available. In therapy trials, monotherapies with dopamine agonists, opiates, benzodiazepines, carbamazepine, clonidine or the combined administration of laevodopa (L-DOPA) in conjunction with a dopadecarboxylase inhibitor have had mixed degrees of success. Most studies have been done on the use of L-DOPA in RLS. In long-term therapy there is a significant alleviation of the complaint, with an improvement in the quality of life and sleep. The disadvantage of the L-DOPA therapy, however, is that in many patients the effectiveness declines and/or there is a shift of the RLS problems to the morning (rebound) or afternoon (augmentation).

For individual dopamine agonists short-term therapy trials have been conducted. The dopamine agonists investigated include: bromocryptine, cabergoline, alphadihydroergocryptine, lisuride, pergolide, pramipexole and ropinirol. All these dopamine agonists were found to be effective. The results of trials on long-term therapy with dopamine agonists are not yet available, so the question of the loss of activity after long-term use (tachyphylaxis) cannot be answered yet.

The disadvantage of the dopamine agonists is the incidence of side-effects such as nausea, vomiting, dizziness, hypotension, constipation and sleeplessness, which generally occur initially and in dose-dependent manner. The use of the anti-Parkinson's drug pramipexole, (S)-2-amino-6-n-propylamino-4,5,6,7-tetrahydro-benzothiazole, a D2/D3 agonist (dopamine agonist), for treating RLS is described in WO 98/31362, to which reference is hereby made in its entirety.

Benzodiazepines and opiates are also effective in RLS. Because of the risk of dependency and the build-up of tolerance, however, these substances are only available for therapy on a restricted basis.

Carbamazepine has only been tested on RLS in a few partly open trials. It gives only partial relief from the complaint and is not currently viewed as a suitable drug for treating RLS.

The effect of clonidine, 2-(2,6-dichloroanilino)-4,5-dihydroimidazole, which was originally developed as an antihypertensive and miotic, in the treatment of RLS has been studied in 4 open trials, 2 double-blind, placebo-controlled trials and a single case study. The daily doses were between 0.1-0.9 mg. The patients reported a (statistically significant) reduction in perceived symptoms such as paresthesia, compulsive movement and tiredness during the day. According to the objective polysomnographic measuring parameters, the sleep latency was indeed shortened, but the quality of sleep, frequency of waking or periodic leg movements in sleep (PLMS) were not affected. Since substances are available which are more effective as monotherapies, clonidine is currently only recommended as an alternative form of therapy under certain circumstances.

A further disadvantage of most monotherapies is that the quantity of the active substance in question has to be increased over time in order to ensure therapeutic success.

### SUMMARY OF THE INVENTION

Surprisingly, it has now been found that the combined administration of clonidine or the hydrochloride thereof together with pramipexole or the hydrochloride thereof leads to an unexpected synergistic effect in terms of suppressing the symptoms of RLS. In fact, it has been found that in combination each of the two active substances can be used in a significantly lower dose that when they are used in monotherapy. In combination therapy a more significant improvement in the condition of the RLS patient is achieved within a short time than was achieved by the relevant monotherapy, even if the latter was carried out over a lengthy period and with fairly high doses.

### DETAILED DESCRIPTION OF THE INVENTION

The present invention provides, as its first aspect, a novel method for the treatment of Restless Leg Syndrome which comprises administering both clonidine or a pharmaceutically acceptable salt thereof and pramipexole or a pharmaceutically acceptable salt thereof. As a second aspect, the invention provides a novel pharmaceutical composition suitable for the treatment of Restless Leg Syndrome which comprises both clonidine or a pharmaceutically acceptable salt thereof and pramipexole or a pharmaceutically acceptable salt thereof.

One advantage of the invention is that the combined administration of clonidine synergistically influences the effect of the dopamine agonist pramipexole (or vice versa) by increasing the activity, so that even low doses of the two active substances are enough to improve the patient's comfort without any intolerable side-effects occurring. In addition, the combined administration of pramipexole with clonidine leads to better responses and a higher response rate in patients with RLS. To what extent the additional administration of clonidine can reverse any tachyphylaxis which might have occurred with therapeutic agents is not yet known, but there is a suspicion of it.

Preferably, the two active substances, clonidine and pramipexole, are used as the hydrochloride. However, other pharmacologically acceptable salts or the neutral compounds may be used. For the active substance combination according to the invention, however, it is not necessary to use both active substances in the form of a salt, especially the same salt (e.g. the hydrochloride). The two active substances may also be used both as neutral compounds and as two different salts or as a combination of a salt of one active substance and the neutral form of the other active substance.

The combination of active substances according to the invention may be formulated according to the current pharmaceutical methods known from the prior art so that they can be administered by oral, spinal, anal or intravenous route or by inhalation, subcutaneously or transdermally. Oral and transdermal preparations are preferred.

The preparation may be given orally in the form of a tablet, powder, powder in a capsule (e.g. a hard gelatine capsule), as a solution or suspension. For spinal, intravenous and subcutaneous applications, the combination of active substances according to the invention is given as a solution. The preparation may be administered anally in suppositories. For inhalation, the combination of active substances may be given as a powder, as an aqueous or aqueous-ethanolic solution or using a propellant gas formulation. For transdermal administration the active substance may be applied to the skin as an ointment or cream, but is preferably applied by means of a plaster.

In the case of plasters, the active substance or combination of active substances is either released directly onto the outer layer of the skin or is released directly into the underlying layers of the skin using a transdermal plaster, in the form of a solution or a gel, e.g. embedded in a polymer matrix, through micro-pins or micro-cutters which penetrate the horny layer of the skin. A transdermal plaster with micro-pins or micro-cutters of this kind is disclosed for example in patent application WO 97/03718. Patent application WO 91/07998 describes a process by means of which active substances can be applied more satisfactorily transdermally by adjusting the skin to a specific pH. U.S. Pat. No. 5,112,842, or the corresponding European Patent EP 0428038, discloses a transdermal plaster for administering pramipexole. Reference is hereby made expressly to the contents of all three patents, to show how the combination of active substances according to the invention can be applied using a transdermal plaster.

Both types of plaster described above (with and without microcutters or micropins) release the active substance continuously onto or into the skin, so as to avoid concentration peaks and the possible side effects associated with them. The active substance or combination of active substances can be released passively or actively. Active transfer can be by purely mechanical means, electrically, osmotically or by iontophoresis. If desired, the release may be controlled electronically, optionally with monitoring of the blood plasma level by sensors or microsensors which are integrated in the plaster or communicate therewith, as a result of which the blood plasma level can be adjusted deliberately to suit individual requirements and consequently a steady release is not absolutely essential.

In every case, the two active substances may be formulated separately (e.g. in a capsule or as a tablet), in a single formulation but separate from one another (e.g. in a capsule with two or more chambers) or mixed together in a single formulation (e.g. in the form of a tablet or in a capsule with only one chamber).

If the two active substances are formulated independently of each other, the two formulations may be supplied in a combined pack (kit).

It is not essential for the two substances to be administered by the same route of administration; rather, combinations of formulations may be used wherein the two active substances are administered by separate routes. For example, clonidine may be given orally while pramipexole is administered transdermally, e.g. using the transdermal plaster described above. However, those formulations wherein the two active substances are administered by the same route are preferred. The two active substances are advantageously administered together in one preparation.

In the case of the transdermal plasters, the two active substances may be administered, for example, either in separate plasters, in a joint plaster in which the two active substances are stored separately within the plaster, or they may be mixed together in one plaster. The same is also true of the other administration forms described above.

The active substance formulation according to the invention is prepared by the methods known from the prior art, depending on the method of administration, and may accordingly contain the formulation constituents known in the art. They may also contain other pharmacologically active substances or cosmetic additives.

Independently of the method of administration, the active substances are preferably administered simultaneously or within an overlapping time frame. In the case of oral administration they should be taken within 1 hour, preferably within 15 minutes of each other.

The amount of clonidine or the pharmacologically acceptable salt of the formulation according to the invention per single dose, in relation to clonidine, corresponds to an oral administration of 0.01 to 1.0 mg, preferably 0.05 to 0.5 mg and most preferably 0.075 to 0.3 mg.

The amount of pramipexole or the pharmacologically acceptable salt thereof, per single dose, corresponds to an oral administration of 0.05 to 2.0 mg, preferably 0.08 to 1.0 mg and most preferably 0.088 to 0.7 mg, based on the neutral compound.

For transdermal use, because of the continuous method of administration, a different quantity may be given to achieve a correspondingly effective blood plasma concentration. The exact amount of active substances can be determined by simple tests, depending on the method of administration.

### EXAMPLE

2 patients with RLS (55 year old man and 67 year old woman) were treated with a combination therapy of pramipexole and clonidine.

1. Therapeutic History: Both patients had been suffering from severe sleep disorders for more than 15 years and had previously been treated with L-DOPA, benzodiazepines (brotizolam, oxazepam), carbamazepine and bromocryptine or pergolide. The symptoms (discomfort, cramps and pains in the legs, compulsive movement, problems falling asleep and sleeping through, as well as daytime tiredness and feelings of exhaustion) improved significantly, but the two patients were never free from symptoms. In both patients, L-DOPA led to typical augmentation during the day which disappeared when they switched to a dopamine agonist. It was not possible to increase the dose of pergolide or bromocryptine any further because of side effects such as nausea, gastrointestinal problems and dizziness. Brotizolam and oxazepam improved the falling asleep and sleeping through, in particular, but these two substances could only be prescribed for a limited time on account of the risk of dependency. After the previous therapy had been brought slowly and completely to an end the two patients were treated with pramipexole in an amount of 0.088 mg two hours before bedtime. In the male patient, the daily dose had to be increased to 0.36 mg at weekly intervals, whilst in the female patient it had to be increased to 0.27 mg. The symptoms certainly improved in both patients, but the two patients did not report any difference from their earlier therapy. The pramipexole was slowly reduced in both patients and finally stopped and a therapy trial with clonidine was started. The clonidine was also initially prescribed in a single dose of 0.075 mg two hours before bedtime and increased by 0.075 mg at intervals of 3 days. The male patient was finally given 0.225 mg, the female patient 0.45 mg of clonidine hydrochloride as a single dose before bedtime; both patients stated that they felt hardly any paresthesia and the compulsive movements had also improved, but the quality of sleep and the number of times they woke during the night had not changed. As a result of some intolerable side effects such as dry mouth, dizziness and constipation, both patients asked if they could stop taking the clonidine.

2. Treatment with a Combination Therapy of Clonidine and Pramipexole After slowly bringing the clonidine therapy to a complete halt and after a treatment-free period of about 1 week, both patients were treated with a combination of 0.088 mg of pramipexole and 0.075 mg of clonidine. From the very first night, both patients reported a significant alleviation of their symptoms. After 7 days the dosage of pramipexole had been increased to 0.18 mg and the dosage of clonidine to 0.15 mg, two hours before going to sleep. At the end of the ₂nd week of treatment, both patients reported that virtually all their subjective symptoms such as tingling, cramp, pain in the legs, restlessness of the legs during the night, problems on going to sleep and sleeping through were no longer present or had been reduced to a tolerable minimum, so that their daily quality of life was no longer impaired. The combined administration of pramipexole and clonidine showed no reduction in activity in either patient right to the end of the observation period of about 3 months.

What is claimed is:

1. A pharmaceutical composition, suitable for treating Restless Leg Syndrome, comprising clonidine or a pharmacologically acceptable salt thereof and pramipexole or a pharmacologically acceptable salt thereof.

2. A pharmaceutical composition according to claim 1, wherein the quantity of clonidine or the pharmacologically acceptable salt thereof per single dose based on clonidine corresponds to an oral dose of 0.01-1.0 mg.

3. A pharmaceutical composition according to claim 1, wherein the quantity of pramipexole or the pharmacologically acceptable salt thereof per single dose based on the neutral compound corresponds to an oral dose of 0.05-2 mg.

4. A method for treating Restless Leg Syndrome, which method comprises administering a therapeutic amount of clonidine or a pharmacologically acceptable salt thereof and pramipexole or a pharmacologically acceptable salt thereof.

5. The method according to claim 4, wherein the quantity of clonidine or the pharmacologically acceptable salt thereof administered, per single dose based on clonidine, corresponds to an oral dose of 0.01-1.0 mg.

6. The method according to according to claim 1, wherein the quantity of pramipexole or the pharmacologically acceptable salt thereof administerd, per single dose based on the neutral compound, corresponds to an oral dose of 0.05-2 mg.
